(19) United States
(12) Patent Application Publication
(10) Pub. No.: US 2010/0285118 A1
(43) Pub. Date: Nov. 11, 2010

(54) ENCAPSULATED OMEGA-3 FATTY ACIDS AND/OR OMEGA-6 FATTY ACIDS AND/OR ESTERS THEREOF WITH A COATING

(76) Inventors: Rob Dekker, AT Vlaardingen (NL); Henk Husken, AT Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/738,327

(22) PCT Filed: Oct. 9, 2008
(86) PCT No.: PCT/EP08/63527

§ 371 Date: May 18, 2010

(30) Foreign Application Priority Data
Oct. 17, 2007 (EP) 07118694.4

(51) Int. Cl. A61K 9/48; A61K 31/355; A61K 31/51; A61K 31/714; A61K 31/352; A61K 31/525; A61K 38/43; A61Q 19/00; A61Q 19/08; B29C 47/04

(52) U.S. Cl. 424/456; 424/463; 514/458; 514/276; 514/52; 514/456; 514/251; 424/94.1; 264/241

(57) ABSTRACT

Encapsulated omega-3 fatty acids and/or omega-6 fatty acids and/or esters thereof with a coating comprising isoflavones, as well as a process to make such by coating encapsulated omega-3 fatty acids and/or omega-6 fatty acids and/or esters thereof with a coating comprising isoflavones.

### FIELD OF THE INVENTION

The present invention relates to an edible product comprising encapsulated omega-3 fatty acids and/or omega-6 fatty acids and/or esters thereof with a coating comprising isoflavones, a process to manufacture such as well as the use of such product.

### BACKGROUND OF THE INVENTION

Specific lipids, oils, and other fatty materials are frequently consumed as part of a health-improving composition. Examples of such compositions are omega-3 fatty acids and/or omega-6 fatty acids and/or esters thereof. Omega-3 fatty acids and/or omega-6 fatty acids are polyunsaturated fatty acids having the unsaturated bonds at a particular position, which contributes to a real and/or perceived effect on human health and/or appearance (e.g. skin condition and/or skin appearance), which has extensively been described in scientific literature and patent publications.

Of such omega-3 fatty acids and/or omega-6 fatty acids and/or esters thereof, said fatty acids preferably have 18-30 carbon atoms and contain at least three double bonds, more preferably 20-24 carbon atoms and contain at least three double bonds. These omega-3 fatty acids and/or omega-6 fatty acids can be in the form of free fatty acid, C1 to C6 alkyl esters thereof, glycerides (including mono-, di- and tri-glycerides, including di- and tri-glycerides wherein omega-3 fatty acids and/or omega-6 fatty acids are present in the di- or tri-glyceride with other fatty acids which may not necessarily be omega-3 fatty acids and/or omega-6 fatty acids) thereof, phospholipid esters thereof or mixtures thereof, all of which are herein collectively referred to as “omega-3 fatty acids and/or omega-6 fatty acids and/or esters thereof”. Such omega-3 fatty acids and/or omega-6 fatty acids and/or esters thereof can be obtained e.g. from marine oils such as fish oils, fish liver oils, and algae, and some specific vegetable oils like borage. Preferred omega-3 fatty acids and/or omega-6 fatty acids (and their derivatives as set out above for the omega-3 fatty acids and/or omega-6 fatty acids) in the present invention comprise DHA (docosahexaenoic acid) and/or EPA (eicosapentaenoic acid), and/or esters thereof.

For some purposes, e.g. for a skin-care-by-ingestion product, it may be preferred that daily an amount of such omega-3 fatty acids and/or omega-6 fatty acids and/or esters thereof is consumed of about 200 mg to 5 g, more preferably between 400 mg and 4 g, even more preferably 400 to 1000 mg. This would be the amount of pure omega-3 fatty acids and/or omega-6 fatty acids and/or esters thereof desired, but since such fatty acids and/or esters thereof are usually commercially offered as part of a (fatty) composition (e.g. fish oil, or algae oil, e.g. in a level of 20 to 40%) the total amount of such composition to be ingested daily is much higher. E.g. when wishing to consume a portion of 660 mg triglycerides of omega-3 fatty acids and/or omega-6 fatty acids, using fish oil with a degree of EPA and DHA taken together of 35%, such skin-care-by-ingestion product should contain about 1.9 g of such fish oil, as daily dose.

Another group of compounds that is reported to have beneficial effects for certain aspects of health and/or appearance and/or condition of e.g. the human skin are isoflavones. Isoflavones in the present invention preferably are compounds having a 3-phenyl-4H-1-benzopyr-4-one backbone, and more preferably such comprises isoflavones (including aglycon forms) and/or derivates thereof, like β-glucoside-, acetyl- and malonyl-methylated- and glycoside-forms. References describing such are e.g. Song et al. 1999. Journal of Agricultural and Food Chemistry 47:1607-1610; Kudou et al. 1991. Agricultural and Biological Chemistry 55:2227-2233; Wang et al. 1998. Journal of the American Oil Chemists Society 75:337-341; and Choi and Rhee. J. of Med Food 9 (1) 2006, 1-10. Isoflavones can e.g. be found in soy, clover sprouts and a number of legumes like chick peas, and products (including extracts and concentrates) derived from these. All the above forms are herein collectively referred to as “isoflavones”. Even more preferred isoflavones herein comprise formononetin, biochanin A, daidzein, genistein and glycitein (formononetin and biochanin A are the methylated forms of daidzein and genistein), daidzin, genistin, glycitin, and/or mixtures thereof.

For some purposes, e.g. for a skin-care-by-ingestion product, it may be preferred that daily an amount of such isoflavones is consumed of about 10-100 mg, preferably about 20-100 mg, more preferably 20-80 mg (when taken as pure isoflavone).

There is information that suggests that for certain purposes related to e.g. improving (or believed to be improving) one or more of health, appearance, skin condition, body functions, or brain functions of humans it can be desired to ingest both omega-3 fatty acids and/or omega-6 fatty acids and/or esters thereof as well as isoflavones. Optionally in addition thereto may be beneficial to be included one or more of carotenoids, vitamins B (e.g. B1, B2, B6, B12), C, or E. Hence, there is a desire for an edible product comprising both omega-3 fatty acids and/or omega-6 fatty acids and/or esters thereof and isoflavones.

WO 2005/077521 discloses a technique for manufacturing coated spherical seamless filled capsules, aimed to disperse a flavouring core upon chewing.

### SUMMARY OF THE INVENTION

Hence, there is a desire for an edible product, e.g. aimed for promoting skin care and/or skin condition by ingestion, comprising omega-3 fatty acids and/or omega-6 fatty acids and/or esters thereof (e.g. in amounts of like 0.4-5 g when calculated as triglyceride ester, per product serving) as well as isoflavones (e.g. in an amount of 1 to 500 mg per product serving). Preferably, such edible product should be in a format which is such that the potential consumer (usually women) would consider it to fit the concept of improving the skin condition (e.g. reducing wrinkles and fine lines) by ingesting actives (possibly referring to omega-3 oils and/or plant oestrogens), and/or that the potential consumer would look forward to taking such edible product when wishing to use a skin-care-by-ingestion product, just by looking at the products. Preferably, the product has the appearance of a food additive, rather than a medicament (as a medicament is believed not fit the concept as well as a food additive).

It is also preferred that such edible product is easy to manufacture (not requiring large investments in equipment and using straightforward ingredients). Preferably the product should be such that it can be manufactured using the isoflavones, carotenoids, vitamins B (e.g. B1, B2, B6, B12), C when present in an particulate form, e.g. like a powder.

It has now been found that the above formulated objectives can be met (at least in part) by an edible product comprising omega-3 fatty acids and/or omega-6 fatty acids and/or esters thereof, said edible product comprising one or more coated capsules, said capsules comprising a core, a capsule wall, and a coating at least partly covering said capsule wall, said core comprising 1-100%, preferably 2-100%, more preferably 10-100% by weight (based on the core) omega-3 fatty acids and/or omega-6 fatty acids and/or esters thereof, and said coating comprising isoflavones.

In the above, it may be preferred that such isoflavones are present in the coating on the capsules in an amount of 0.01-20% of the weight of the core, preferably in an amount of 0.02-10%, more preferably 0.5-10%.

It was also found that such can be prepared a process for the preparation of a coated capsule, said coated capsule comprising a core comprising 1-100%, preferably 2-100%, more preferably 10-100% omega-3 fatty acids and/or omega-6 fatty acids and/or esters thereof, a capsule wall, and a coating at least partly covering said capsule wall on the outside of said capsule wall, said coating comprising isoflavones, said process comprising the steps of:
providing a capsule comprising a core comprising 1-100%, preferably 2-100%, more preferably 10-100% by weight (based on the core) omega-3 fatty acids and/or omega-6 fatty acids and/or esters thereof, and a capsule wall,
applying one or more coating layers comprising isoflavones.

In the above process, the edible capsules comprising the omega-3 fatty acids and/or omega-6 fatty acids and/or esters thereof can be made using any suitable technique as known in the art for making edible or ingestible capsules of e.g. a diameter of between 2 and 10 mm. Encapsulating a hydrophobic matter is well documented. A specific technique (which is specific as it yields seamless capsules which, when coated, have specific advantages, e.g. upon chewing by the consumer) is set out in WO 2005/077521, but a capsule comprising 1-100%, preferably 2-100%, more preferably 10-100% by weight (based on the core) omega-3 fatty acids and/or omega-6 fatty acids and/or esters thereof and a capsule wall may be made in any other suitable technique, which is well within the skill of the skilled person.

In the product and process according to this invention, if the core comprises less than 100% of omega-3 fatty acids and/or omega-6 fatty acids and/or esters thereof, it may be preferred that the remainder of the core material comprises a hydrophobic matter, like e.g. triglycerides or fat-soluble matter.

In the present invention, the capsule wall material preferably comprises a gel-forming and film forming protein or polysaccharide biopolymer which is solid at room temperature.

### DETAILED DESCRIPTION OF THE INVENTION

The compositions according to the invention comprise omega-3 fatty acids and/or omega-6 fatty acids and/or esters thereof, and of such omega-3 fatty acids and/or omega-6 fatty acids and/or esters thereof, said fatty acids preferably have 18-30 carbon atoms and contain at least three double bonds, more preferably 20-24 carbon atoms and contain at least three double bonds. These omega-3 fatty acids and/or omega-6 fatty acids can be in the form of free fatty acid, C1 to C6 alkyl esters thereof, glycerides (including mono-, di- and tri-glycerides, including di- and tri-glycerides wherein omega-3 fatty acids and/or omega-6 fatty acids are present in the di- or tri-glyceride next to other fatty acids which may not necessarily be omega-3 fatty acids and/or omega-6 fatty acids) thereof, phospholipid esters thereof or mixtures thereof, all of which are herein collectively referred to as “omega-3 fatty acids and/or omega-6 fatty acids and/or esters thereof”. Such omega-3 fatty acids and/or omega-6 fatty acids and/or esters thereof can be obtained e.g. from marine oils such as fish oils, fish liver oils, and algae, and some specific vegetable oils like borage. Preferred omega-3 fatty acids and/or omega-6 fatty acids (and their derivatives as set out above for the omega-3 fatty acids and/or omega-6 fatty acids) in the present invention comprise DHA (docosahexaenoic acid) and/or EPA (eicosapentaenoic acid), and/or esters thereof. It is also to be understood herein, that whenever an amount of omega-3 fatty acids and/or omega-6 fatty acids and/or esters thereof is referred to, such amount is to be calculated as if it were triacylglycerides.

Isoflavones in the present invention preferably are compounds having a 3-phenyl-4H-1-benzopyr-4-one backbone, and more preferably such comprises isoflavones (including aglycon forms) and/or derivates thereof, like β-glucoside-, acetyl- and malonyl-methylated- and glycoside-forms. References describing such are e.g. Song et al. 1999. Journal of Agricultural and Food Chemistry 47:1607-1610; Kudou et al. 1991. Agricultural and Biological Chemistry 55:2227-2233; Wang et al. 1998. Journal of the American Oil Chemists Society 75: 337-341; and Choi and Rhee. J. of Med Food 9 (1) 2006, 1-10. Isoflavones can e.g. be found in soy, clover sprouts and a number of legumes like chick peas, and products (including extracts and concentrates) derived from these. All the above forms are herein collectively referred to as “isoflavones”. Even more preferred isoflavones herein comprise formononetin, biochanin A, daidzein, genistein and glycitein (formononetin and biochanin A are the methylated forms of daidzein and genistein), daidzin, genistin, glycitin, and/or mixtures thereof.

When it is referred to herein as “vitamin C”, it is to be understood as to comprise also one or more of its salts and precursors, such as ascorbic acid and salts of ascorbic acid and/or sources thereof.

When it is referred to herein as “vitamin B” it is to be understood as to comprise one or more of the various members of the family of vitamin B, like B1, B2, B6 and B12, and/or precursors thereof and/or sources thereof.

When it is referred to herein as “vitamin E”, it is to be understood as to comprise also one or more of various forms of vitamin E, like tocopheryl acetate, and more specifically d-α-tocopheryl acetate, as well as sources of these compounds.

When it is referred to herein as “carotenoids”, it is to be understood as to comprise one or more of lycopene, beta-carotene, lutein, astaxanthin, zeaxanthin, alpha-carotene, beta-cryptocanthin, canthaxanthin. It also covers sources of carotenoids, in particular when they contain at least 1% of such carotenoids. An example of this is a tomato fraction which is high in lycopene. It also covers carotenoids and/or sources thereof which are mixed with an auxiliary component, such as a carrier like maltodextrin.

The coating process can be performed in any suitable way, with the coating process as set out in WO 2005/077521 being preferred, both for conventional capsules with a seam as well as for the seamless capsules which can be made using the technique as referred to in WO 2005/077521. In more detail: the coating process preferably comprises at least one stage (preferably successive stages) of:
contacting the capsules to be coated with a sticking agent comprising a concentrated aqueous solution of a sugar and/or polyol and/or gum arabicum,
intimately mixing such, e.g. in a panning machine or tumbler, with optionally adding a particulate matter (which may comprise the isoflavones) followed by
a stage in which the so-applied coating layer is allowed to dry.

As said, then this series of three steps may be repeated until all the desired isoflavones are in the coating.

In the above, the amount of the concentrated solution of a sugar and/or polyol and/or gum arabicum is preferably such that it is (per coating stage) between 0.5% and 10% (by weight) of the capsule-weight, preferably between 1% and 5%. In the final product (coated capsule) it may be preferred that the total amount of sugar and/or polyol is between 5 and 70% by weight, based on the final capsule. The total amount of the gum arabicum (is preferably between 1 and 20% by weight, based on the final capsule.

Such coating with a particulate matter can be achieved in a suitable way when the capsules are treating with an agent which can stick or “glue” such particulate matter to the capsule. Applying such sticking agent can be applied to the capsule before the particulate agent is applied, or simultaneously, or afterwards. It is preferred that prior to and/or simultaneously with applying the isoflavone, a sticking agent is applied to the capsules. Preferred sticking agents comprise an aqueous solution of a sugar and/or polyol and/or gel-forming or thickening biopolymer. Most preferred as sticking agent are sugar and/or polyols and/or gum arabicum. Hence, it is preferred that said sticking agent comprises sugar and/or polyol and/or arabicum. Depending on the amount of isoflavone to be adhered to the capsule, it can be desired to go through a plurality of cycles of adding a sticking agent followed by (or simultaneously with or added before) the isoflavone. A preferred polyol here comprises maltitol.

As a first layer on the still uncoated capsule, especially in the case of a capsule wall comprising a substantial portion of gelatin, it is preferred that at least the first coating layer on the capsule wall comprises at least one layer comprising gum arabicum, either added as solution, as particulate powder or both. Such may lead to better coating, e.g. due to roughening of the surface.

When applying multiple coating steps, in between coating steps, it may be desired that one waits with applying a next coating until the previous one is fully dry (dry as one may achieve with air at otherwise ambient conditions, so some residual moisture may retain). This is in particular preferred when adding a plurality of coating layers of sugar and/or polyol. Adding such plurality (e.g. 5-50) layers of sugar and/or polyol and/or fat and/or chocolate (which are preferably solid at room temperature) may be done to achieve sufficient mechanical robustness.

Regarding the capsule comprising the core comprising 1-100%, preferably 2-100%, more preferably 10-100% omega-3 fatty acids and/or omega-6 fatty acids and/or esters thereof, such can be prepared using conventional techniques for encapsulating a hydrophobic matter, e.g. a technique which is used for commercial capsules of the desired matter, such as for example complex coacervation, extrusion, melt injection, and coextrusion. Also, commercial fish oil capsules may be used for coating in this respect (e.g. soft capsules), in particular when their capsule wall comprises gelatin, preferably in an amount of 60-100%. In an embodiment which may be preferred (or is preferred when one wishes to make high quality chewable capsules) the manufacture of the still uncoated capsules may be along the lines as set out in e.g. WO 2005/077521 (i.e. using submerged coextrusion).

More specifically, providing the capsule wall may preferably be achieved by coextruding the core material and the capsule wall material through a coextrusion nozzle, the coextrusion nozzle being immersed in an oil bath of a temperature well below the gel point of the gel-forming protein or polysaccharide biopolymer of the capsule wall material: preferably at least 10° C., more preferably at least 20° C. below its gel point, the core material being extruded through the inner nozzle and the (aqueous solution of) capsule wall material through the outer nozzle of the coextrusion nozzle. Needless to say the feed of the capsule wall material (preferably an aqueous solution comprising 60-100% gelatin (by weight on the uncoated capsule) is preferably kept above the T_{gel} of said gel-forming protein or polysaccharide biopolymer of the capsule wall material. When obtaining the fresh capsules, and before they are completely dry (the capsule wall that is) they are preferably kept in motion, to avoid sticking together. In order to facilitate formation of individual capsules, it is preferred in the above process that the coextrusion nozzle is vibrated with a vibration of between 1 and 100 Hz, preferably between 5 and 50 Hz.

In the edible product of the present invention, the amount of omega-3 fatty acids and/or omega-6 fatty acids and/or esters thereof in said coated capsules, calculated as triacylglycerides per capsule is preferably between 5 mg and 5 g. The wide range is both dependent on the dosing one would like to give the consumer, but also in how many coated capsules such is to be achieved. Preferred actual dosings (of one complete serving) of this compound are between 200 mg and 5 g, more preferably between 400 mg and 4 g. The number of capsules over which it is to be distributed is preferably between 1 and 100, more preferably between 2 and 50, even more preferably between 3 and 50, and most preferably between 10 and 50. Hence, it may be preferred that the edible product of the present invention is a portioned product comprising per portion between 1 and 100, preferably between 2 and 50, more preferably between 3 and 50, and most preferably between 10 and 50 of coated edible capsules herein disclosed. Preferably, the edible product consists of the coated capsules herein disclosed (in the numbers indicated above per dosing). Per capsule the preferred amount of omega-3 fatty acids and/or omega-6 fatty acids and/or esters thereof is 5 to 1000 mg, more preferably between 10 and 200 mg, most preferably between 15 and 100 mg. All these amounts refer to omega-3 fatty acids and/or omega-6 fatty acids and/or esters thereof in a pure form. Usually, these are commercially applied as a fatty mixture containing such omega-3 fatty acids and/or omega-6 fatty acids and/or esters thereof, for example a fish oil containing 30 or 40% of these omega-3 fatty acids and/or omega-6 fatty acids and/or esters thereof, the remainder mostly being triglycerides of other fatty acids. Consequently, the total amount of fatty matter (e.g. triglycerides) in the edible composition and coated capsules according to the present invention thus is proportionally higher.

Likewise the amount of isoflavone per capsule in the edible product according to the present invention may show wide variation, e.g. between 0.05 mg and 500 mg, more preferably between 0.1 mg and 100 mg, and even more preferably between 1 and 20 mg per capsule. The total amount of isoflavone per serving size (for number of coated capsules per serving size see above) is preferably between 10 and 100 mg, more preferably between 20 and 80 mg. Per capsule the preferred amount of isoflavone would be preferably between 0.1 and 100 mg, more preferably between 0.5 and 20 mg. Again, this refers to the amount of pure isoflavone. Isoflavone is frequently offered as a preparation containing e.g. 60, 70 or 80% isoflavone, and hence the total matter of isoflavone-containing compound in the edible composition and coated capsules according to the present invention thus is proportionally higher.

The edible product according to the present invention is preferably a portioned product, comprising per portion, more preferably consisting per portion, between 1 and 100, preferably between 2 and 50, more preferably between 3 and 50, most preferably between 10 and 50 of said coated edible capsules. Such portioned product can e.g. be offered as a food additive, e.g. a skin-care-by-ingestion food additive. It is preferred that a portioned product is packaged such that each portion is in one single container (e.g. a pot, jar, bottle, blister pack), as such allows dosing the desired amount easily.

For certain health effects or benefits, or effects on skin condition and/or appearance (real or perceived), it may be preferred that the omega-3 fatty acids and/or omega-6 fatty acids and/or esters thereof and the isoflavones are present in a certain ratio or relative amount to each other. Hence, it may be preferred that, if isoflavone is present, for said coated capsule in the edible product according to the present invention the weight ratio of isoflavones:omega-3 fatty acids and/or omega-6 fatty acids and/or esters thereof when calculated as triacylglycerides, is between 1:10000 and 1:5, preferably between 1:1000 and 1:10, more preferably between 1:100 and 1:10.

In the process according to the present invention, the capsules which are to be coated can be provided with any suitable technique that can yield (edible) capsules of omega-3 fatty acids and/or omega-6 fatty acids and/or esters thereof, as long as they can be handled, preferably in a panning machine. For use in the panning machine it is also preferred that the capsule wall is of such a material that it can be treated with a sugar and/or gum arabicum coating layer, preferably when such are applied in a concentrated (e.g. above 20% by weight) of an aqueous solution. Commercially, fish oil capsules are available, which can be subjected to the coating as herein described. Alternatively, a specific technique yielding capsules which are seamless (compared to capsules made by other processes) is set out in WO 2005/077521. Capsules so-prepared have the advantage that upon chewing, they give generally a more favourable mouthfeel than capsules prepared in other ways, e.g. with seams. Hence, it is possible to prepare the capsules to be coated with the process as set out in WO 2005/077521, and such is in fact preferred. This document also sets out the requirement to the materials, e.g. the capsule wall material.

In the present invention, the capsule wall material of the capsules in the edible product preferably comprises a gel-forming and film forming protein or polysaccharide biopolymer which is solid at room temperature. Preferred biopolymers in this respect are: gelatin, alginate, calcium alginate, agar, guar gum, carrageenan, starch mixed with pectin, gelatin mixed with pectin; in an amount of 60-100%, preferably in an amount of 70-90%, by weight based on the (dry) capsule wall. The remainder may be e.g. a plasticiser. A typical plasticiser is glycerol. Gelatin is the most preferred capsule wall herein.

Regarding the coated capsules of the edible product according to the invention, it is preferred that the coating comprises a plurality of layers, comprising one or more layers comprising gum arabicum. Also, it may be preferred that the coating comprises a plurality of layers, comprising one or more layers comprising a sugar and/or polyol which sugar and polyol are solid when at 25° C. and in dry form.

As mentioned before, the process preferably allows coating using a sticking agent. Preferably, when coating with isoflavone, it may be preferred that the coating after having been applied, comprises isoflavone, and that it further contains a sugar and/or polyol and/or gum arabicum and/or fat and/or chocolate, e.g. as sticking agent, preferably in such an amount that the weight ratio isoflavone:sugar and/or polyol and/or gum arabicum and/or fat and/or chocolate in the same layer is between 1:0.1 and 1:200. Suitable polyols herein include maltitol and/or xylitol, and preferably the coating comprises a mixture of xylitol with maltitol, e.g. in a ratio of xylitol:maltitol of 10:1 to 1:10, and/or sugar.

The coated capsules of the edible product according to the present invention may also comprise additional components such as vitamin E, vegetable oil, and some flavourings. Such components may be applied both in the core as well as in the coating layers. Hence, it may be preferred that the capsule wall or coating further comprises one or more of minerals, vitamin B, vitamin C, carotenoids, flavourings, colourings.

The coated edible capsules of the edible composition may further comprise vitamin C. When the coated capsule according to the present invention comprises vitamin C, it is preferred that for said coated capsule the weight ratio of vitamin C:omega-3 fatty acids and/or omega-6 fatty acids and/or esters thereof when calculated as triacylglycerides, is between 1:200 and 1:0.1, preferably between 1:50 and 1:1. When a vitamin C is present, it is preferred that such is present in an amount of between 50 and 1000 mg, when calculated as pure vitamin C, preferably between 100 and 500 mg (per serving size: so per capsule it is to be divided by the number of capsules per serving, which is preferably between 1 and 100). Per capsule the preferred amount of vitamin C (when present) would be preferably between 0.2 and 50 mg, more preferably between 1 and 10 mg.

The coated edible capsules of the edible composition may further comprise a carotenoid. When the coated capsule according to the present invention comprises a carotenoid (or more carotenoids) it is preferred that for said coated capsule the weight ratio of carotenoid:omega-3 fatty acids and/or omega-6 fatty acids and/or esters thereof when calculated as triacylglycerides, is between 1:10000 and 1:50, more preferably between 1:500 and 1:100. When a carotenoid is present, it is preferred that such is present in an amount of between 1 and 10 mg, when calculated as pure carotenoid, preferably between 2 and 8 mg (per serving size: so per capsule it is to be divided by the number of capsules per serving, which is preferably between 1 and 100). Per capsule the preferred amount of carotenoid (when present) would be preferably between 0.01 and 2 mg, more preferably between 0.02 and 0.5 mg.

As said before, the technique to make seamless capsules may be the preferred (for e.g. mouthfeel) technique to create the capsules to be coated, in particular when capsules are to be made which are intended to be chewed rather than swallowed as a whole. In that case, the coating is preferably also a seamless coating (which can be achieved using the technique as set out above).

Preferably, for robustness of the capsules, the capsules are spherical. Spherical herein as expressed by a ratio between the largest diameter and the smallest diameter being not more than 1.3. However, when commercial fish oil capsules are taken to be coated, due to the manner of preparation, they are usually more oval in shape. Out of convenience (availability) such shape may thus also be preferred.

Although a wide variety of sizes can be manufactured it is preferred that the coated capsules each have a weight of 0.02-5 g, more preferably 0.1-1 g. The coating may weight between 10 and 300% of the uncoated capsule. Similarly, it may be preferred that the coated capsules each have a diameter of 2 to 15 mm, preferably 3 to 10 mm.

In the present invention, the coating-free capsules preferably have a capsule wall thickness of 5-400 micron, and a diameter of the coating-free capsule of 1-15, preferably 3-7 mm, and a wall thickness to capsule diameter ratio in the range of 0.005 to 0.05 (when dry).

The invention further relates to the use of an edible product according to this invention, as a skin care by ingestion product. The invention furthermore relates to such use by women, preferably by post-menopausal women. The invention also relates to either or both of such uses to reduce (the visibility of) fine lines and/or wrinkles and/or age spots.

### EXAMPLES

### Example 1

Gelatin capsules were prepared of a commercial fish oil (ONC Unimeg-38, containing 38% of EPA and DHA taken together, with added thereto vitamin E as alpha-tocopheryl acetate) by the following process.

A capsule wall solution was prepared by dissolving pig gelatin (280 Bloom, Symrise) in water at pH 5.2 (concentration 25%) and glycerol as plasticiser by heating to 80° C., and subsequent cooling to 45° C.

The fish oil and the gelatin solution were fed through a submerged coextrusion nozzle (dimension inner nozzle 1 mm), with the fish oil in the inner nozzle and the gelatin solution in the outer nozzle. The feed line for the gelatin solution was kept at 60-80° C. The coextrusion nozzle was connected with an oscillator, hitting the nozzle with a frequency of around 13.8 Hz. The nozzle was located submerged in a bath filled with liquid vegetable oil, which was held at a temperature of approx. 14° C.

The flow was set at approx. 55 ml/min for the fish oil, and about 19 ml/min for the gelatin solution. Due to the oscillation of the nozzle, the liquids broke up into individual droplets offish oil, coated with a layer of gelatin solution. Due to the immersion in the cool oil bath, the gelatin solidifies to form a coating layer.

The nozzle was adjusted to give capsules of fish oil with a gelatin wall of approx. 5.5 mm diameter, which corresponded with a weight of approx. 80-85 mg (size and weight when still wet).

The so-prepared encapsulated fish oil droplets were removed from the oil by sieving. Subsequently, the capsules were placed in a refrigerator for 1 hour at about 4° C. Following this, adhering oil was removed by centrifuging for 30 seconds at 600 rpm. To reduce stickiness, about 1% by weight (based on the capsules) of SiO₂ was added (it was found that about 0.2-0.3% of said SiO₂ remained on the capsules).

The so-prepared capsules were dried by air in a tumbling dryer (18 rpm) with air of a temperature of about 16° C., for 2 hours. The so-obtained capsules were stable to handle.

Subsequently, the so-obtained capsules (2 kg when uncoated) were subjected to a coating process (batch process), along the following steps:
coating (twice) with a base layer of sugar and gum arabicum, to roughen the surface of the gelatin and thus to facilitate further coating. To this end, two aqueous solutions were prepared, one containing 40% (wt) gum arabicum, the other 72% (wt) sugar, which were then mixed in 48% both and 4% water added. Per coating layer 30 ml of said mix solution was added to the capsules in the panning machine, followed by dusting with a dry mixture of gum arabicum (about 20%) and sugar (about 80%), with 10 minute drying after each such coating;
coating with 10 sugar layers. To this end about 20-30 ml of a sugar solution (72% wt) was added to the capsules;
coating with 3 layers of a mixture containing isoflavone (Novasoy 70, ex ADM), vitamin C and lycopene (tomato-derived, 10% on a carrier). To this end, the capsules were first treated with a layer of sugar and gum arabicum (30 ml of an aqueous solution containing 40% wt sugar and 72% wt gum arabicum) as adhesive, after which the capsules were sprayed with a dry particulate mixture of (wt %) isoflavones (10%), vitamin C (35%) and lycopene (6%, Tomat-O-Red 10% CWD, ex Lycored), sugar (24.5%), gum arabicum (24.5%) which was then repeated a further 2 times to give 3 layers;
coating with 30 layers of sugar, in the same way as the second coating step (sugar layers);
coating with a gloss-providing layer (Quick Glanz H, containing shellac and carnauba wax) and
coating with a lacquer layer (Quick Lac, containing shellac), and a colouring layer (dispersed in alcohol) providing colour.

All the above coating layers were applied in a panning machine with the pan rotating at about 30 rpm under an angle of about 20°, per batch of about 2 kg, at ambient conditions. During coating, air was blown through the panning machine, of ambient temperature. For the second series of coating (the 10 sugar layers) sufficient time was allowed to enable complete drying (and thus crystallisation) of the sugar. The lacquer layer is applied by spraying.

The sugar-coating layers applied gave additional robustness, e.g. to facilitate packaging and processing, but such can be omitted or reduced in number of layers.

The resulting coated capsules had an appearance of spherical capsules, having an overall composition as below, and the overall weight had increased from 2 kg per batch to about 5.5 kg per batch.

TABLE 1
overall composition example 1.

| | Amount in |
|---|---|
| Ingredient | weight % |
| Sugar | 40.2% |
| Fish oil | 32.8% |
| Sugar/gum arabicum mixture | 15.5% |
| Vitamin C | 4.1% |
| Isoflavone | 1.1% |
| Gelatin | 2.9% |
| Glycerol | 0.7% |
| Vitamin E | 0.6% |
| Lycopene (Tomat-O-Red 10% CWD (Lycored) | 0.5% |
| Minors (colourings, flavourings, sucralose, gloss | balance |
| and laquer coating, pigments, residual moisture) | |

The resulting capsules combined in one physically stable product omega-3 fatty acids and/or omega-6 fatty acids and/or esters thereof (in this case as fish oil) in one product with isoflavones, vitamin C, lycopene of natural origin and vitamin E. For a desired level of intake of the fish oil, isoflavones, vitamins E and C, and lycopene a serving size can be a fixed number of such spherical capsules, e.g. 10, 20, 30, 40, or 50 depending on the total dosing desired.

### Example 2

Uncoated capsules were made in the same way as in example 1, and with the same composition as in example 1. These capsules were coated in a similar fashion as in example 1. The coating composition for example 2 differed from example 1 in that for example 2 no lycopene and vitamin C were present in the coating layers, and that instead of the sugar a combination of maltitol and xylitol was used.

The coating of the capsules (2 kg when uncoated) was along the following steps:
coating with a base layer of maltitol and gum arabicum, to roughen the surface of the gelatin and thus to facilitate further coating. To this end, an aqueous solution was prepared containing gum arabicum and maltitol (total solids 50%, of which 10% gum arabic and 40% maltitol). About 39 g of said mix solution was added to the capsules in the panning machine, followed by dusting with 125 g Quick Coat Maltitol (ex A. Wolf), with 10 minute drying after each such coating; coating with 15 maltitol layers. To this end a solution of maltitol (65% wt) and gum arabic (3%) was added to the capsules, per layer about 40-45 g, with air drying at 25-30° C.;
coating with 3 layers of a mixture containing isoflavone. To this end, the capsules were coated in 3 layers: first spraying about 25 g of a solution, then applying about 120 g of a particulate mixture of isoflavone (18%) and Quick Coat (based on maltitol, ex A. Wolf, 82%), followed by one sealing layer of 40 g solution and 360 g Quick Coat Xylitol (ex A. Wolf), with air drying in between each coating layer;
coating with 38 layers of xylitol. This was done by using a composition (about 30 ml per layer) containing 71% Xylisorb 700 (ex Roquette), 4% gum arabic solution 40% and 25% water. Every 2-4 layers air drying was applied in between the layers.
Coating with 7 layers (about 40 g per layer) of a composition containing xylitol (68%), titan dioxide (1%) and colouring (2%) in water, to provide colour;
coating with 2 layers of a gloss-providing layer (Quick Glanz H, containing shellac and carnauba wax)
coating with a lacquer layer (Quick Lac, containing shellac), and a colouring layer (dispersed in alcohol) layer providing colour.

The resulting overall composition was similar as in the table in example 1, except for replacing sugar with a combination of xylitol and maltitol (about three-quarters xylitol, one quarter maltitol), and the absence of vitamin C and lycopene.

The overall appearance was similar to the coated capsules as prepared following example 1.

### Example 3

A panel of 10 woman aged 20-65 were explained in written text in a few sentences the concept of improving the skin condition (reducing wrinkles and fine lines) by ingesting actives, referring to omega-3 oils and plant oestrogens.

The woman were then presented two product formats in measured portions, containing both about 1.8 g fish oil (containing about 37% DHA/EPA) and 40 mg soy isoflavones (Novasoy 70):
a cup containing 32 coated capsules prepared following example 2 (“caps” in table 2 below).
a cup containing about 1.8 g fish oil and dispersed therein about 40 mg soy isoflavones (“liq.” In table below).

The woman were subsequently asked, when seeing both samples simultaneously, to give a score for each format on the following 3 questions on a scale 1 to 10:1 meaning “not at all” (i.e. negative), and 10 “extremely” (i.e. very positive):
Q1: just by looking at the products, how well do you think that they fit the concept that was shown to you?
Q2: just by looking at the products, how much do you look forward to taking them?
Q3: just by looking at the products, how easy do you think they are to take every day?

The results on said scale were set out in table 2.

TABLE 2

| | | 1 | 2 | 3 | 4 | 5 | 6 | 7 | 8 | 9 | 10 | mean |
|---|---|---|---|---|---|---|---|---|---|---|---|---|
| Q1 | Caps. | 8 | 10 | 8 | 8 | 8 | 7 | 5 | 7 | 9 | 9 | 8 |
| | Liq. | 1 | 1 | 2 | 6 | 4 | 4 | 6 | 3 | 2 | 5 | 3 |
| Q2 | Caps. | 8 | 8 | 9 | 7 | 9 | 8 | 1 | 3 | 7 | 5 | 7 |
| | Liq. | 1 | 1 | 2 | 5 | 4 | 4 | 10 | 2 | 1 | 3 | 3 |
| Q3 | Caps. | 10 | 8 | 8 | 7 | 10 | 6 | 1 | 5 | 5 | 1 | 6 |
| | Liq. | 9 | 1 | 8 | 9 | 8 | 5 | 10 | 4 | 8 | 7 | 7 |

### Example 4

Gelatin capsules (not yet coated) as described in example 1 were coated with a coating comprising chocolate.

For this, the so-obtained capsules (2 kg when uncoated) were subjected to a coating process (batch process), along the following steps:
coating with a base layer of gum arabicum and isomalt (to roughen the surface of the gelatin and thus to facilitate further coating). To this end, two aqueous solutions were prepared, one containing 40% (wt) gum arabicum, the other 65% (wt) isomalt, which were then mixed with water (25% gum arabicum, 13.5% water, and 61.5% isomalt solution). A coating layer of 40 g of said mix solution was added to the capsules in the panning machine, followed by dusting with 125 g of a dry mixture of gum arabic (20%) and isomalt (80%), followed by 10 minute drying after such coating;
coating with 15 layers containing Isomalt GS (ex Palatinit GmbH). To this end for each step about 35 ml of a solution of about 64% Isomalt GS and 4% gum arabicum was added to the capsules, followed by air drying after each step;
preparation of a chocolate-active mixture. By mixing and roller-refining (below 18 micron): Maltisorb P200 (maltitol) 46%, cocoa mass 13%, cocoa butter 16%, whole milk powder 25%. 90% of this mixture was conched (7 hours) with 9.5% cocoabutter and 0.5% lecithin. A mixture was prepared containing 58% of the above described maltitol-chocolate, 14.5% sodium ascorbate, 4.5% ascorbic acid, 5% isoflavones, 2.5% Tomat-O-Red 10% CWD, 15% cocoabutter, 0.5% flavour. On a batch of 2000 g uncoated capsules 1325 g of the above chocolate-active mixture was coated in several steps until the complete mixture was taken up.
Coating with an intermediate coating of gum arabicum and isomalt, in the same way as the first coating step;
coating with 30 layers of Isomalt GS, in the same way as the second coating step (sugar layers);
coating with a gloss-providing layer (Quick Glanz H, containing shellac and carnauba wax) and
coating with a lacquer layer (Quick Lac, containing shellac), and a colouring layer (dispersed in alcohol) providing colour.

All the above coating layers were applied in a panning machine with the pan rotating at about 30 rpm under an angle of about 20°, per batch of about 2 kg.

### Example 5

In the same way as for example 4, coated capsules were made which contained instead of the conched and roller-refined chocolate a mixture of: 2200 g Maltisorb P200, 650 g cocoabutter (which was roller-refined) and added thereto 425 g cocoabutter and 14 g lecithin.

1. An edible product comprising omega-3 fatty acids and/or omega-6 fatty acids and/or esters thereof, said edible product comprising one or more coated edible capsules, said capsules comprising a core, a capsule wall, and a coating at least partly covering said capsule wall, said core comprising 1-100%, by weight (based on the core) omega-3 fatty acids and/or omega-6 fatty acids and/or esters thereof, said coating comprising isoflavones, in amount of between 0.1 mg and 100 mg per coated capsule, wherein the coated capsules each have a weight of 0.02-5 g.

2. The edible product according to claim 1 wherein the amount of omega-3 fatty acids and/or omega-6 fatty acids and/or esters thereof in said coated capsules, calculated as triacylglycerides per capsule is between 5 mg and 1000 mg.

3. The edible product according to claim 1 wherein said core comprises 2-100%, by weight, based on the core, omega-3 fatty acids and/or omega-6 fatty acids and/or esters thereof.

4. The edible product according to claim 1 wherein the amount of isoflavone per coated capsule is between 0.5 and 20 mg.

5. The edible product according to claim 1 wherein for said coated capsule the weight ratio of isoflavones:omega-3 fatty acids and/or omega-6 fatty acids and/or esters thereof when calculated as triacylglycerides, is between 1:10000 and 1:5.

6. The edible product according to claim 1 wherein said coated capsule wall comprises a gel-forming and film forming biopolymer, said biopolymer comprising one of gelatin, alginate, calcium alginate, agar, guar gum, carrageenan, starch mixed with pectin, in an amount of 60-100%, by weight based on the capsule wall.

7. The edible product according to claim 1 wherein the coating comprises a plurality of layers, comprising one or more layers comprising of a sugar and/or polyol and/or fat and/or chocolate which are solid when at 25° C. and in dry form.

8. The edible product according to claim 1 wherein the coating comprising isoflavone further contains a sugar and/or polyol and/or gum arabicum and/or fat and/or chocolate, preferably in such an amount that the weight ratio isoflavone:sugar and/or polyol and/or gum arabicum and/or fat and/or chocolate in the same layer is between 1:0.1 and 1:200.

9. The edible product according to claim 1 wherein the core further comprises one or more of: carotenoids, vitamin E, vegetable oil, flavourings.

10. The edible product according to claim 1 wherein the capsule wall or coating further comprises one or more of minerals, vitamin B, vitamin C, carotenoids, flavourings, colourings.

11. The edible product according to claim 1 characterised in that the coated capsules each have a diameter of 2 to 15 mm.

12. The edible product according to claim 1 wherein the edible product is a portioned product, comprising per portion between 1 and 100 of said coated edible capsules.

13. A process for the preparation of a coated capsule, said coated capsule comprising a core comprising 1-100% omega-3 fatty acids and/or omega-6 fatty acids and/or esters thereof, a capsule wall, and a coating at least partly covering said capsule wall on the outside of said capsule wall, said coating comprising isoflavones, said process comprising the steps of:
providing a capsule comprising a core comprising 1-100% by weight (based on the core) omega-3 fatty acids and/or omega-6 fatty acids and/or esters thereof, and a capsule wall,
applying one or more coating layers comprising isoflavones.

14. The process according to claim 13, wherein providing the capsule is achieved by coextruding the core material and the capsule wall material through a coextrusion nozzle, the coextrusion nozzle being immersed in an oil bath of a temperature at least 10° C. below the gel point of the film-forming protein or polysaccharide biopolymer of the capsule wall, the core material being extruded through the inner nozzle and the capsule wall material through the outer nozzle of the coextrusion nozzle.

15. A method for skin care by ingestion, the method comprising ingesting an edible product according to claim 1.

16. The method according to claim 15, wherein the ingestion is by women.

17. The method according to claim 16 wherein the method reduces fine lines and/or wrinkles and/or age spots.
